(19) United States
(12) Patent Application Publication
(10) Pub. No.: US 2025/0000601 A1
(43) Pub. Date: Jan. 2, 2025

(54) Assistive Medical Accessory

(71) Applicant: Boston Scientific Scimed, Inc., Maple Grove, MN (US)

(72) Inventors: Judith Tiferes Wang, Kenton, OH (US); Kristi Mae Flury, Maple Grove, MN (US)

(73) Assignee: Boston Scientific Scimed, Inc., Maple Grove, MN (US)

(21) Appl. No.: 18/758,523

(22) Filed: Jun. 28, 2024

Related U.S. Application Data
(60) Provisional application No. 63/524,554, filed on Jun. 30, 2023.

(51) Int. Cl. A61B 46/10; A61B 90/00

(52) U.S. Cl. CPC A61B 46/10; A61B 2090/0807

(57) ABSTRACT

Assistive medical accessories and methods for making and using assistive medical accessories are disclosed. An example assistive medical accessory may include a sterile drape configured for use with a medical device. The sterile drape may have a top surface. A visual indicator may be disposed along the top surface. The visual indicator may be configured to provide use information to a clinician using the medical device.

### CROSS-REFERENCE TO RELATED APPLICATIONS

This application claims the benefit of priority under 35 U.S.C. § 119 of U.S. Provisional Application No. 63/524,554, filed Jun. 30, 2023, the entire disclosure of which is hereby incorporated by reference.

### TECHNICAL FIELD

The present disclosure pertains to medical devices. More particularly, the present disclosure pertains to assistive medical accessories including, for example, sterile drapes.

### BACKGROUND

A wide variety of medical devices have been developed for medical use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### BRIEF SUMMARY

This disclosure provides design, material, manufacturing method, and use alternatives for medical devices. An assistive medical accessory is disclosed. The assistive medical accessory comprises: a sterile drape configured for use with a medical device; wherein the sterile drape has a top surface; and a visual indicator disposed along the top surface, the visual indicator being configured to provide use information to a clinician using the medical device.

Alternatively or additionally to any of the embodiments above, the visual indicator includes text.

Alternatively or additionally to any of the embodiments above, the visual indicator includes one or more graphics.

Alternatively or additionally to any of the embodiments above, the visual indicator includes one or more icons.

Alternatively or additionally to any of the embodiments above, the visual indicator includes printed instructions for use of the medical device that include text, graphics, or a combination of text and graphics.

Alternatively or additionally to any of the embodiments above, the visual indicator includes one or more check boxes.

Alternatively or additionally to any of the embodiments above, the visual indicator includes one or more set of grid lines.

Alternatively or additionally to any of the embodiments above, the visual indicator includes a shadow board.

A medical device system is disclosed. The medical device system comprises: a packaged medical device; and a sterile drape accessory for use with the packaged medical device, the sterile drape accessory having a visual indicator printed thereon, the visual indicator being configured to assist a clinician in using the packaged medical device.

Alternatively or additionally to any of the embodiments above, the visual indicator includes text.

Alternatively or additionally to any of the embodiments above, the visual indicator includes one or more graphics.

Alternatively or additionally to any of the embodiments above, the visual indicator includes one or more icons.

Alternatively or additionally to any of the embodiments above, the visual indicator includes printed instructions for use of the packaged medical device that include text, graphics, or a combination of text and graphics.

Alternatively or additionally to any of the embodiments above, the visual indicator includes one or more check boxes.

Alternatively or additionally to any of the embodiments above, the visual indicator includes one or more set of grid lines.

Alternatively or additionally to any of the embodiments above, the visual indicator includes a shadow board.

A method for performing a medical procedure is disclosed. The method comprises: disposing a sterile drape along a work surface of a clinical facility; wherein the sterile drape has a visual indicator printed thereon; observing the visual indicator to gain knowledge sufficient to assist a clinician in using a medical device; and performing a medical procedure using the medical device.

Alternatively or additionally to any of the embodiments above, the visual indicator includes instructions for use of the medical device, and wherein performing a medical procedure using the medical device includes performing the medical procedure according to the instructions for use.

Alternatively or additionally to any of the embodiments above, the instructions for use of the medical device that include text, graphics, or a combination of text and graphics.

Alternatively or additionally to any of the embodiments above, the visual indicator includes a set of grid lines, and further comprising arranging one or more medical accessories along the set of grid lines.

Alternatively or additionally to any of the embodiments above, the visual indicator includes a shadow box, and further comprising disposing a medical accessory adjacent to the shadow box.

Alternatively or additionally to any of the embodiments above, the visual indicator includes one or more check boxes, wherein performing a medical procedure using the medical device includes performing a plurality of steps, and further comprising using a marking device to check at least one of the one or more check boxes upon completion of one of the plurality of steps.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a schematic overview of an example catherization lab.

FIG. 2 is a schematic overview of an example back table.

FIG. 3 illustrates an example assistive medical accessory and a packaged medical device disposed on an example back table.

FIG. 4 illustrates an example assistive medical accessory.

FIG. 5 illustrates an example assistive medical accessory.

FIG. 6 illustrates an example assistive medical accessory.

FIG. 7 illustrates an example assistive medical accessory.

FIG. 8 illustrates an example assistive medical accessory.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the invention.

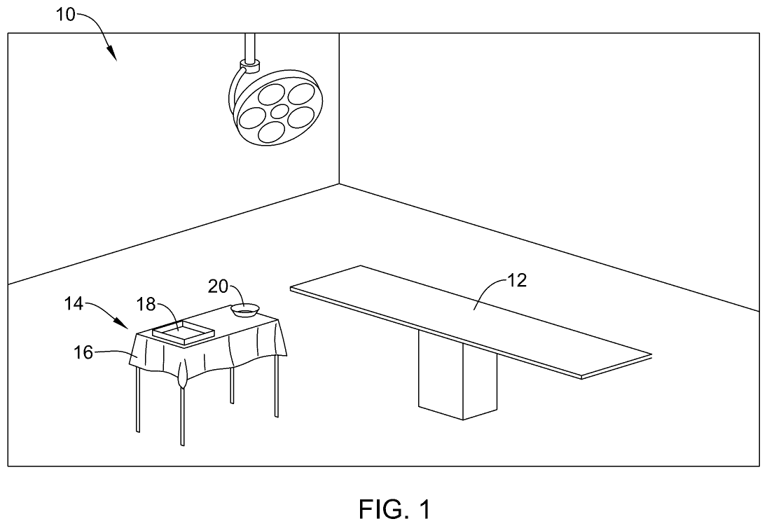

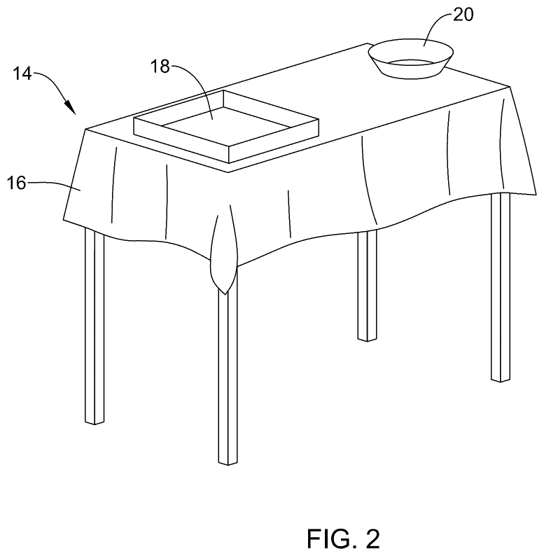

The use of drapes in a medical/surgical setting are commonplace and help to reduce the spread of infection, among other things. Drapes may be placed along work surfaces (e.g., tables, benches, etc.), in operating rooms, in catheterization laboratories, along or adjacent patients, and/or in any suitable location. For example, FIG. 1 schematically illustrates a clinical setting such as a catheterization laboratory 10 having a patient table 12 and a back table 14. A sterile drape 16 may be disposed on, for example, the back table 14. One or more medical devices and/or accessories 18, 20 may be disposed on the sterile drape 16. It can be appreciated that the setting may vary. FIG. 2 is a more detailed view of the example back table 14 and the sterile drape 16 disposed thereon. Here it can be seen that the accessory 18 may take the form of a tray and accessory 20 may take the form of a bath. A variety of different accessories are contemplated. In some of these and in other instances, a medical device (e.g., a catheter, delivery system, guidewire, and/or the like) may be disposed on the sterile drape 16.

Some medical interventions may require several steps to prepare and/or use one or more medical devices. In some of these and in other instances, it may be desirable to have additional guidance and/or instruction for using the one or more medical devices. In some of these and in other instances, it may be desirable to organize devices/accessories for use during an intervention. Disclosed herein are assistive medical accessories that include one or more visual indicators disposed thereon (e.g., along a top surface thereof) that aid a clinician. The assistive medical accessories may include sterile drapes with a visual indictor disposed thereon that can help aid a clinician.

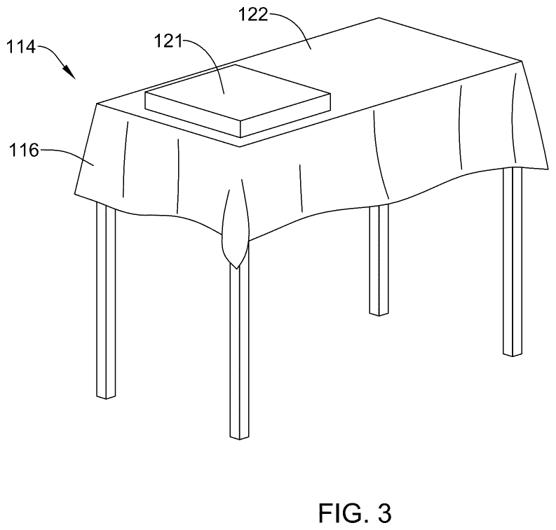

FIG. 3 illustrates another example table or substrate 114 having an assistive medical accessory 116 disposed thereon. In this example, the assistive medical accessory 116 may take the form of a sterile drape 116 and/or a sterile drape accessory 116. The sterile drape 116 may have a top surface 122. A medical device 121 may be disposed on the top surface 122. In this example, the medical device 121 may be a packaged medical device. In use, the sterile drape 116 may be paired with the packaged medical device 121 so that visual indicators (discussed herein) may be tailored to aid a clinician with using the packaged medical device 121. For example, if the packaged medical device 121 takes the form of a medical device delivery system (e.g., a stent delivery system, a prosthetic heart valve delivery system, etc.), the sterile drape 116 may include one or more visual indicators configured to convey use information for the packaged medical device 121. Accordingly, such visual indicators may aid a clinician with using the packaged medical device 121.

In one example, the packaged medical device 121 may take the form of a prosthetic heart valve and corresponding delivery system. It can be appreciated that delivering the prosthetic heart valve may include several steps such as loading the prosthetic valve into/onto the delivery system, flushing, navigating the prosthetic valve through the anatomy to a target site, and/or deploying the prosthetic valve. In some instances, these processes may include many steps, some of which may be time consuming and highly detailed. The use of printed instructions 126 on the sterile drape 116 may help to make sure a clinician is following appropriate steps/procedures. In addition, the printed instructions 126 on the sterile drape 116 may serve as a reminder to a clinician of what steps/procedures are required and/or a reminder of how to perform such steps/procedures. This is just one example. Other devices/systems are contemplated.

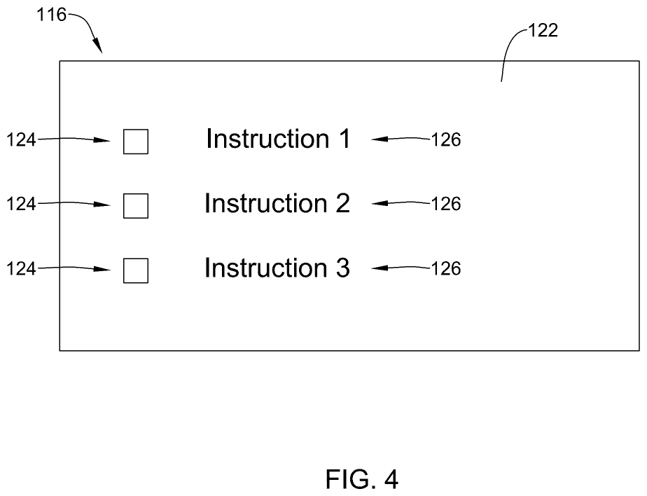

FIG. 4 schematically illustrates the top surface 122 of the sterile drape 116. Here it can be seen that the sterile drape 116 may include visual indicators in the form of printed instructions 126. For example, sequential instructions for use of the medical device 121 are represented in FIG. 4 using the text “Instruction 1”, “Instruction 2”, and “Instruction 3”, where each of the instructions are intended to represent text and/or graphics that convey instructions for using a medical device (e.g., the packaged medical device 121 as shown in FIG. 3). The content of the printed instructions 126 can vary. For example, the printed instructions 126 can mirror or mimic the instructions for use that accompany the packaged medical device 121. In some of these and in other instances, the printed instructions 126 may include a summarized or simplified version of the instructions for use, which may highlight certain aspects of the instructions for use that may be complicated, cumbersome, or otherwise deserving of emphasis.

The printed instructions 126 may include printed text. The text may include one or more colors, fonts, or text type (e.g., including, for example, bolding, underlining, italicizing, increased font size, decreased font size, etc.). For example, if the printed instructions 126 contain a warning or contraindication, the printed instructions 126 may include an emphasis in the form of a colored text (e.g., red text), an enlarged text, bolded and/or underlined text, etc. In some of these an in other instances, the printed instructions 126 may include graphics, pictures, icons, and/or the like that may help to convey use information. For example, the printed instructions 126 may include graphics that depict the proper use and/or the proper execution of a portion of a given intervention. The graphics may be highly schematic and/or cartoonish in design, highly detailed and/or otherwise including technically accurate depictions of devices and/or steps, combinations thereof, and/or fall within the range of highly schematic to highly technical.

In at least some instances, the printed instructions 126 may be disposed on or otherwise printed on the top surface 122 of the sterile drape 116 prior to sterilization of the sterile drape 116. For example, the desired printed instructions 126 may be printed onto the sterile drape 116 using a sterilizable ink or printing substance. Thereafter, the sterile drape 116 can be sterilized. The use of the sterilizable ink allows for the printed instructions 126 to be retained on the sterile drape 116 after a suitable sterilization procedure. In some of these and/or other instances, the printed instructions 126 may be activated upon sterilization of the sterile drape 116. For example, the printed instructions 126 may be applied to the top surface 122 of the sterile drape 116 as autoclave indictor tape (and/or similar) so that the text of the printed instructions 126 become visible upon sterilization.

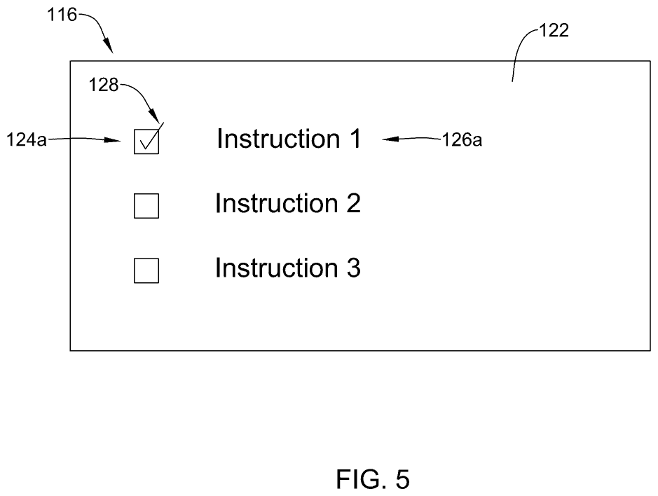

In some instances, additional or alternative visual indicator may be disposed on the top surface 122 of the sterile drape 116. For example, FIG. 4 illustrates a series of check boxes 124. In this example, the check boxes 124 may be positioned adjacent to the printed instructions 126. This may allow, for example, a clinician to use a suitable marking device (e.g., such as a permanent marker) to put a completed check mark 128 in the appropriate check box 124 as shown in FIG. 5. This may help a clinician to track the progress of various preparation and/or treatment steps during a given intervention. It can be appreciated that a clinician may put additional completed check marks 128 in other ones of the check boxes 124 at suitable times during an intervention to track progress.

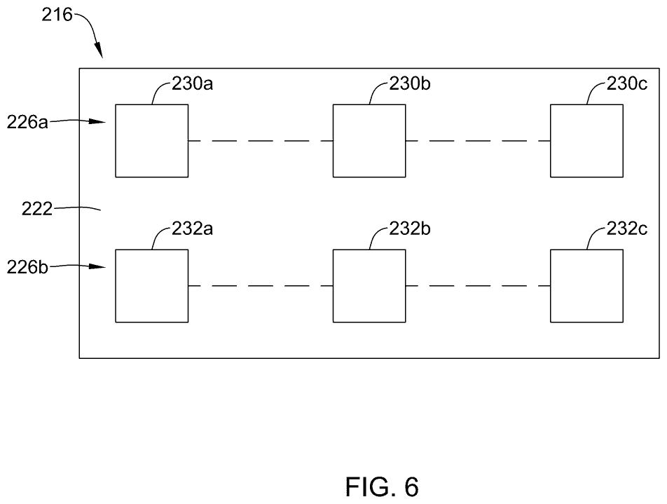

FIGS. 6-8 illustrate some additional example visual indicators that may be used in addition to the those shown in FIGS. 4-5 and/or as alternative visual indicators to those shown in FIGS. 4-5. For example, FIG. 6 depicts a first set of printed instructions 226a printed onto a top surface 222 of a sterile drape 216 (e.g., an assistive medical accessory 216 taking the form of a sterile drape 216). The first set of printed instructions 226a may include separately conveyed text or graphics, as schematically shown at 230a, 230b, 230c. A second set of instructions 226b may be printed on the top surface 222. The second set of instructions 226b may include separately conveyed text or graphics, as schematically shown at 232a, 232b, 232c. It can be appreciated that the boxes numbered as 230a, 230b, 230c and 232a, 232b, 232c, as shown in FIG. 6, may be understood represent graphics, pictures, icons, and/or the like. The use of the boxes numbered as 230a, 230b, 230c and 232a, 232b, 232c is merely exemplary as a wide variety of different graphics can be used without departing from the spirit of the disclosure. Thus, FIGS. 4-5 can be seen as illustrating text for the printed instructions 126 (e.g., while also describing and/or otherwise it being understood that the printed instructions 126 can include printed text, graphic, or combinations thereof), and FIG. 6 can be seen as schematically illustrating graphics for printed instructions 226, 226b (e.g., while also describing and/or otherwise it being understood that the printed instructions 226, 226b can include printed text, graphics, or combinations thereof). In addition, FIG. 6 may also be understood to illustrate that the printed instructions 226a, 226b arranged in rows. Other arrangements are contemplated.

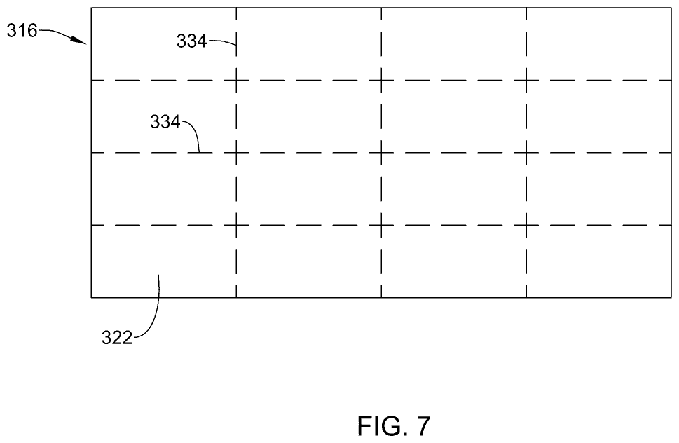

FIG. 7 illustrates another example sterile drape 316 (e.g., an assistive medical accessory 316 taking the form of a sterile drape 316) similar in form and function to other drapes disclosed herein. The sterile drape 316 may have a top surface 322. A set of grid lines 334 may be disposed along the top surface 322. The grid lines 334 may help a clinician to arrange devices thereon in a manner that is organized and useful for a given intervention. In other words, the grid lines 334 may serve to aid a clinician in arranging devices along the sterile drape 316 in a manner that is useful to the clinician. It can be appreciated that the grid lines 334 may be used alone on a sterile drape 316 or in conjunction with any of the other visual indicators disclosed herein.

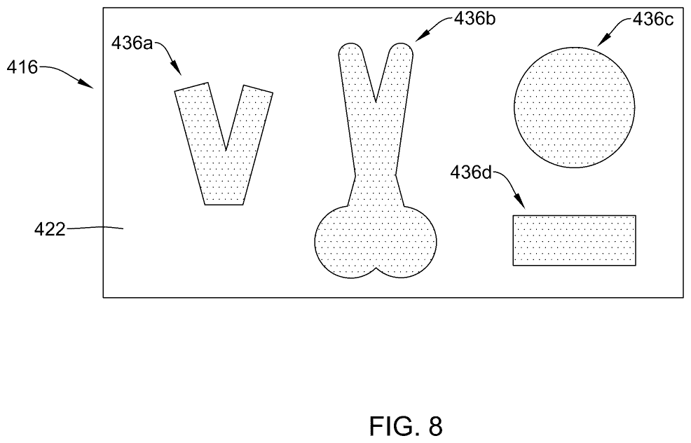

FIG. 8 illustrates another example sterile drape 416 (e.g., an assistive medical accessory 416 taking the form of a sterile drape 416) similar in form and function to other drapes disclosed herein. The sterile drape 416 may have a top surface 422. One or more shadow boards such as shadow boards 436a, 436b, 436c, 436d may be disposed along the top surface 422. The shadow boards 436a, 436b, 436c, 436d may provide a schematic representation resembling the shape of an instrument or device that may be used for a particular intervention. This visual indicator may provide a marked location suitable for placing a device thereon. For example, the shadow board 436a may represent a forceps such that the shadow board 436a serves as a visual indicator of a potential location for placing a forceps. It can be appreciated that the shadow boards that may be similar in form and function to the shadow boards 436a, 436b, 436c, 436d may be used alone on a sterile drape 416 or in conjunction with any of the other visual indicators disclosed herein.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. An assistive medical accessory, comprising:
a sterile drape configured for use with a medical device;
wherein the sterile drape has a top surface; and
a visual indicator disposed along the top surface, the visual indicator being configured to provide use information to a clinician using the medical device.

2. The assistive medical accessory of claim 1, wherein the visual indicator includes text.

3. The assistive medical accessory of claim 1, wherein the visual indicator includes one or more graphics.

4. The assistive medical accessory of claim 1, wherein the visual indicator includes printed instructions for use of the medical device that include text, graphics, or a combination of text and graphics.

5. The assistive medical accessory of claim 1, wherein the visual indicator includes one or more check boxes.

6. The assistive medical accessory of claim 1, wherein the visual indicator includes one or more set of grid lines.

7. The assistive medical accessory of claim 1, wherein the visual indicator includes a shadow board.

8. A medical device system, comprising:
a packaged medical device; and
a sterile drape accessory for use with the packaged medical device, the sterile drape accessory having a visual indicator printed thereon, the visual indicator being configured to assist a clinician in using the packaged medical device.

9. The medical device system of claim 8, wherein the visual indicator includes text.

10. The medical device system of claim 8, wherein the visual indicator includes one or more graphics.

11. The medical device system of claim 8, wherein the visual indicator includes printed instructions for use of the packaged medical device that include text, graphics, or a combination of text and graphics.

12. The medical device system of claim 8, wherein the visual indicator includes one or more check boxes.

13. The medical device system of claim 8, wherein the visual indicator includes one or more set of grid lines.

14. The medical device system of claim 8, wherein the visual indicator includes a shadow board.

15. A method for performing a medical procedure, the method comprising:
disposing a sterile drape along a work surface of a clinical facility;
wherein the sterile drape has a visual indicator printed thereon;
observing the visual indicator to gain knowledge sufficient to assist a clinician in using a medical device; and
performing a medical procedure using the medical device.

16. The method of claim 15, wherein the visual indicator includes instructions for use of the medical device, and wherein performing a medical procedure using the medical device includes performing the medical procedure according to the instructions for use.

17. The method of claim 16, wherein the instructions for use of the medical device that include text, graphics, or a combination of text and graphics.

18. The method of claim 15, wherein the visual indicator includes a set of grid lines, and further comprising arranging one or more medical accessories along the set of grid lines.

19. The method of claim 15, wherein the visual indicator includes a shadow box, and further comprising disposing a medical accessory adjacent to the shadow box.

20. The method of claim 15, wherein the visual indicator includes one or more check boxes, wherein performing a medical procedure using the medical device includes performing a plurality of steps, and further comprising using a marking device to check at least one of the one or more check boxes upon completion of one of the plurality of steps.
